(19) United States
(12) Patent Application Publication
(10) Pub. No.: US 2025/0084034 A1
(43) Pub. Date: Mar. 13, 2025

(54) IMMUNOSUPPRESSANT FORMULATIONS

(71) Applicant: Novartis AG, Basel (CH)

(72) Inventors: Philippe Michel Rene BOUILLOT, Basel (CH); Emeric REYNAUD, Village Neuf (FR)

(21) Appl. No.: 18/802,089

(22) Filed: Aug. 13, 2024

Related U.S. Application Data
(63) Continuation of application No. 17/230,175, filed on Apr. 14, 2021, now Pat. No. 12,071,402.
Application No. 17/230,175 is a continuation of application No. 16/804,926, filed on Feb. 28, 2020, now abandoned.
Application No. 16/804,926 is a continuation of application No. 13/917,730, filed on Jun. 14, 2013, now abandoned.
Application No. 13/917,730 is a continuation-in-part of application No. PCT/EP2012/050151, filed on Jan. 5, 2012.

(30) Foreign Application Priority Data
Jan. 7, 2011 (EP) 11150431.2

(51) Int. Cl. C07D 205/04; A61K 9/20; A61K 31/397

(52) U.S. Cl. CPC C07D 205/04; A61K 9/2004; A61K 31/397

(57) ABSTRACT

The present invention relates to a solid phase pharmaceutical composition comprising one or more pharmaceutically acceptable excipients and an active pharmaceutical ingredient (“API”) which is a compound of formula A1 or A2 or a pharmacologically acceptable salt, solvate or hydrate thereof, wherein the API is not exposed to a basic compound.

### FIELD OF THE INVENTION

The present invention relates to formulations of immunosuppressant compounds, and particularly to formulations of S1P receptor modulators. More particularly the invention relates to formulations of 1-{4-[1-(4-cyclohexyl-3-trifluoromethyl-benzyloxyimino)-ethyl]-2-ethyl-benzyl}-azetidine-3-carboxylic acid, pharmaceutically acceptable salts, and related compounds.

### BACKGROUND TO THE INVENTION

S1P receptors belong to a family of closely related, lipid activated G-protein coupled receptors. S1P1, S1P3, S1P2, S1P4, and S1P5 (also respectively termed EDG-1, EDG-3,EDG-5, EDG-6, and EDG-8) are identified as receptors specific for sphingosine-1-phosphate (S1P). Certain S1P receptors are associated with diseases mediated by lymphocyte interactions, for example, in transplantation rejection, autoimmune disease, inflammatory diseases, infectious diseases and cancer.

WO2004/103306 discloses immunosuppressant compounds that are useful in the treatment of diseases associated with S1P receptor mediated signal transduction. The immunosuppressant compounds disclosed in WO2004/103306 affect the pathology and/or symptomology of these diseases by altering the activity of S1P receptors. In particular, WO2004/103306 and US 2009/0036423 disclose 1-{4-[1-(4-cyclohexyl-3-trifluoromethyl-benzyloxyimino)-ethyl]-2-ethyl-benzyl}-azetidine-3-carboxylic acid:

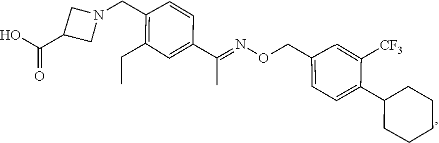 [SMILES: CCc1cc(/C(C)=N/OCC2=CC=C(C3CCCCC3)C(C(F)(F)F)=CC2)ccc1CN1CC(C(=O)O)C1]

and methods for making this compound. The compounds are disclosed as useful in the treatment and/or prevention of diseases and disorders mediated by lymphocyte interactions, for example autoimmune diseases, e.g. rheumatoid arthritis, systemic lupus erythematosus, Hashimoto's thyroiditis and multiple sclerosis, amongst many others. WO2004/103306 and US 2009/0036423 are incorporated herein by reference for all purposes, including by way of

non-limiting example paragraphs [0041]-[0054], inclusive, of US 2009/0036423 and each example thereof.

WO2010/020610 discloses use of S1P receptor agonists, for example 1-{4-[1-(4-cyclohexyl-3-trifluoromethyl-benzyloxyimino)-ethyl]-2-ethyl-benzyl}-azetidine-3-carboxylic acid, in the treatment of peripheral neurapathies, such as Guillain-Barre syndrome (GBS), chronic inflammatory demyelinating polyradiculoneuropathy (CIDP), multifocal motor neuropathy with conduction block (MMN), and paraproteinaemic demyelinating peripheral neuropathy (PDN).

WO2007/021666 discloses a concentrate for dilution comprising a S1P receptor agonist, propylene glycol and optionally glycerin, which concentrate is described as being stable for extended periods of time. One compound mentioned as an S1P receptor agonist is 1-{4-[1-(4-cyclohexyl-3-trifluoromethyl-benzyloxyimino)-ethyl]-2-ethyl-benzyl}-azetidine-3-carboxylic acid. The dilution disclosed in WO2007/021666 is provided in a liquid form and is therefore particularly suitable for patients who struggle to swallow solid medications.

WO2009/048993 discloses compositions comprising S1P receptor modulators, such as 2-substituted 2-amino-propane-1,3-diol or 2-amino-propanol derivatives, which are suitable for use as an oral dosage form. The composition is disclosed to comprise the active ingredient and one or more of various specified excipients. Example 10 mentions glyceryl behenate as a non-feasible excipient, seemingly because of degradation of the active (FTY720). One S1P modulator mentioned is 1-{4-[1-(4-cyclohexyl-3-trifluoromethyl-benzyloxyimino)-ethyl]-2-ethyl-benzyl}-azetidine-3-carboxylic acid.

WO2010/072703 discloses a dosage regimen of an S1P receptor modulator for the treatment of patients suffering from an autoimmune disease, for example multiple sclerosis. The dosage regimen comprises administering a lower dosage of an S1P receptor modulator during the initial days of treatment compared to the standard daily dosage. The dosage is then increased stepwise up to the standard daily dosage of the S1P receptor modulator. One S1P modulator mentioned is 1-{4-[1-(4-cyclohexyl-3-trifluoromethyl-benzyloxyimino)-ethyl]-2-ethyl-benzyl}-azetidine-3-carboxylic acid.

### SUMMARY OF THE INVENTION

The present invention is predicated at least in part on a finding that, in relation to active pharmaceutical ingredients described later in this specification, pharmaceutical compositions having particular excipients are associated with reduced degradation of the active pharmaceutical ingredient as compared to alternative compositions of the same active pharmaceutical ingredient.

The invention is also predicated at least in part by a finding that provision of the active pharmaceutical ingredient as particles of relatively large size can improve stability whilst maintaining an adequately homogeneous content uniformity.

The compounds to which the application relates are compounds as disclosed in WO 04/103306 and US 2009/0036423, in particular compounds of formula A1 or A2:

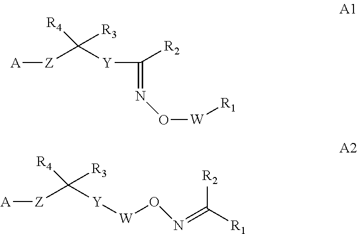

wherein
A is COOR₅, OPO(OR₅)₂, PO(OR₅)₂, SO₂OR₅, POR₅OR₅ or 1H-tetrazol-5-yl, R₅ being H or an ester-forming group, e.g. C₁₋₆alkyl, and in one implementation of the invention being H;
W is a bond, C₁₋₃alkylene or C₂₋₃alkenylene;
Y is C₆₋₁₀aryl or C₃₋₉heteroaryl, optionally substituted by 1 to 3 radicals selected from halogen, NO₂, C₁₋₆alkyl, C₁₋₆alkoxy; halo-substituted C₁₋₆alkyl and halo-substituted C₁₋₆alkoxy;
Z is chosen from:

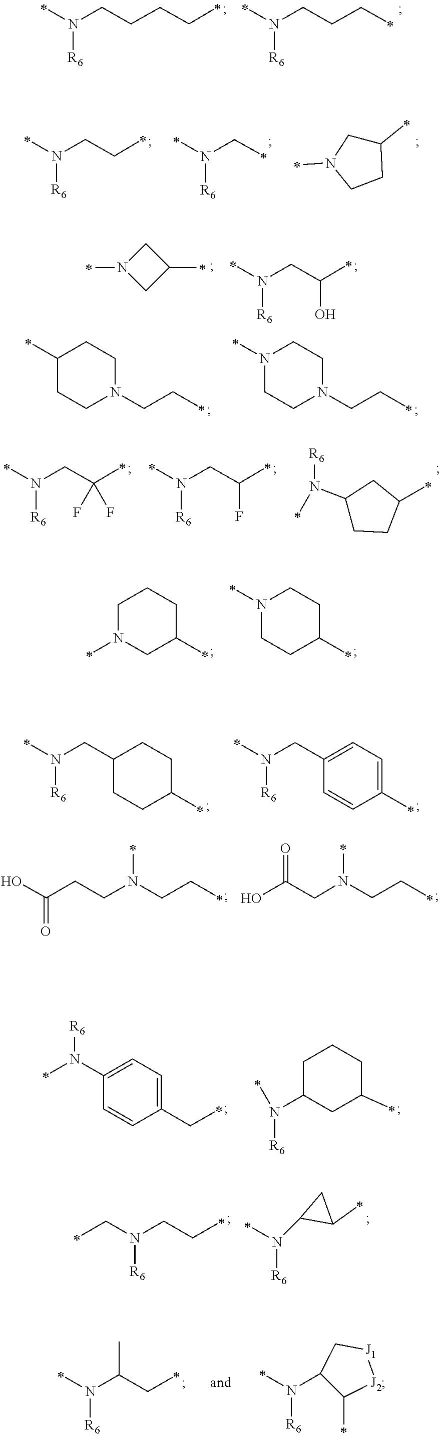

wherein the asterisks of Z indicate the point of attachment between —C(R₃)(R₄)— and A of Formula Ia or Ib, respectively; R₆ is chosen from hydrogen and C₁₋₆alkyl; and J₁ and J₂ are independently methylene or a heteroatom chosen from S, O and NR₅; wherein R₅ is chosen from hydrogen and C₁₋₆alkyl; and any alkylene of Z can be further substituted by one to three radicals chosen from halo, hydroxy, C₁₋₆alkyl; or R₆ can be attached to a carbon atom of Y to form a 5-7 member ring;
R₁ is C₆₋₁₀aryl or C₃₋₉heteroaryl, optionally substituted by C₁₋₆alkyl, C₆₋₁₀aryl, C₆₋₁₀aryIC₁₋₄alkyl, C₃₋₉heteroaryl, C₃₋₉heteroaryIC₁₋₄alkyl, C₃₋₈cycloalkyl, C₃₋₈cycloalkylC₁₋₄alkyl, C₃₋₈heterocycloalkyl or C₃₋₈heterocycloalkylC₁₋₄alkyl; wherein any aryl, heteroaryl, cycloalkyl or heterocycloalkyl of R₁ may be substituted by 1 to 5 groups selected from halogen, C₁₋₆alkyl, C₁₋₆alkoxy and halo substituted-C₁₋₆alkyl or —C₁₋₆alkoxy;
R₂ is H, C₁₋₆alkyl, halo substituted C₁₋₆alkyl, C₂₋₆alkenyl or C₂₋₆alkynyl; and
each of R₃ or R₄, independently, is H, halogen, OH, C₁₋₆alkyl, C₁₋₆alkoxy or halo substituted C₁₋₆alkyl or C₁₋₆alkoxy;
or a pharmacologically acceptable salt, solvate or hydrate thereof.

In the above formula, the designation “C₁₋₆” means “having 1, 2, 3, 4, 5 or 6 carbon atoms” and the designation “C₃₋₈” means “having 3, 4, 5, 6, 7 or 8 carbon atoms”. The designation “C₁₋₄” means “having 1, 2, 3 or 4 carbon atoms”. The designation “C₃₋₉” means “having 3, 4, 5, 6, 7, 8 or 9 carbon atoms”.

The invention particularly, but not exclusively, involves compounds of formula A1 or A2 in which R₅ is H, e.g. moiety A is —COOH, in its acid form (not therefore as a salt thereof).

An exemplary compound of formula A1 or A2 is 1-{4-[1-(4-cyclohexyl-3-trifluoromethyl-benzyloxyimino)-ethyl]-2-ethyl-benzyl}-azetidine-3-carboxylic acid and its pharmaceutically acceptable salts, e.g. its hemifumarate salt.

In one aspect, the invention provides a pharmaceutical composition comprising an active pharmaceutical ingredient (“API”) selected from the compounds of Formulae A1 and A2, and one or more pharmaceutically acceptable excipients, wherein the API is not exposed to a basic compound.

In another aspect, the invention provides a pharmaceutical composition comprising an active pharmaceutical ingredient (“API”) selected from the compounds of Formulae A1 and A2, wherein the API is in the form of particles having an X90 diameter of at least 8 μm and optionally of at least 10 μm.

The invention also provides a pharmaceutical composition comprising an active pharmaceutical ingredient (“API”) selected from the compounds of Formulae A1 and A2, wherein the API is in the form of particles having a crystallinity of 80% or more.

Further included in the invention are pharmaceutical compositions comprising an active pharmaceutical ingredient (“API”) selected from the compounds of Formulae A1 and A2, wherein the API is in the form of particles having an X90 diameter of at least 8 μm and optionally of at least 10 μm and a crystallinity of 80% or more.

The invention includes a pharmaceutical composition comprising an active pharmaceutical ingredient (“API”) selected from the compounds of Formulae A1 and A2, and one or more pharmaceutically acceptable excipients, wherein the API is in the form of particles which have an X90 diameter of at least 8 μm and optionally of at least 10 μm and which are not exposed to a basic compound. In an embodiment, the composition is free of basic compounds.

The invention includes pharmaceutical compositions comprising an active pharmaceutical ingredient (“API”) selected from the compounds of Formulae A1 and A2, and one or more pharmaceutically acceptable excipients, wherein the API is in the form of particles which have a crystallinity of 80% or more and which are not exposed to a basic compound.

Additionally to be mentioned as provided by the invention are pharmaceutical compositions comprising an active pharmaceutical ingredient (“API”) selected from the compounds of Formulae A1 and A2, and one or more pharmaceutically acceptable excipients, wherein the API is in the form of particles which have have an X90 diameter of at least 8 μm and optionally of at least 10 μm and a crystallinity of 80% or more and which are not exposed to a basic compound.

The composition is in particular a solid phase composition, for example a tablet or capsule, particularly a tablet. The composition may be coated with a moisture barrier and an exemplary composition is a tablet coated with a moisture barrier.

The pharmaceutical composition may therefore comprise, or consist of, at least one API and one or more non-basic excipients. The one or more non-basic excipients may be selected from binders, disintegrants, glidants, lubricants, fillers, diluents, and/or sorbents. The tablets may comprise one or more tablet lubricants wherein the lubricants in the tablets are selected exclusively from stearic acid, hydrogenated vegetable oil, mineral oil, polyethylene glycol 4000-6000, gyceryl palmitostearate and glyceryl behenate.

In one embodiment, the pharmaceutical composition comprises a non-basic sustained release agent, for example a non-basic hydrogel former, e.g. a hypromellose (hydroxypropylmethyl cellulose).

A particular embodiment resides in a tablet comprising a compressed mixture which consists of 1-{4-[1-(4-cyclohexyl-3-trifluoromethyl-benzyloxyimino)-ethyl]-2-ethyl-benzyl}-azetidine-3-carboxylic acid or a pharmaceutically acceptable salt thereof e.g. a hemifumarate salt and one or more non-basic excipients. The 1-{4-[1-(4-cyclohexyl-3-trifluoromethyl-benzyloxyimino)-ethyl]-2-ethyl-benzyl}-azetidine-3-carboxylic acid or salt in a particular implementation is in the form of particles which have an X90 diameter of from 10 μm to 200μm and/or which are at least 80% crystalline. The compressed mixture may include a desiccant and/or be coated with a moisture barrier (e.g. both includes a desiccant and have a moisture barrier coating).

The pharmaceutical compositions of the present invention may be used to treat autoimmune diseases, e.g. rheumatoid arthritis, systemic lupus erythematosus, Hashimoto's thyroiditis and multiple sclerosis, amongst many others, e.g. as disclosed in WO2004/103306 and US 2009/0036423 for example in paragraphs [0041]-[0042] of US 2009/0036423. The pharmaceutical compositions of the present invention may be used to treat peripheral neurapathies, for example motor neuron disease, Guillain-Barre syndrome (GBS), chronic inflammatory demyelinating polyradiculoneuropathy (CIDP), multifocal motor neuropathy with conduction block (MMN), or paraproteinaemic demyelinating peripheral neuropathy (PDN). In particular embodiments, the compositions of the invention are used to treat multiple sclerosis. The invention therefore includes a method for treating a subject in need thereof, e.g. having or suspected of having one of the aforesaid diseases, e.g. multiple sclerosis, comprising administering to the patient a composition of the invention. The composition is desirably administered in an effective amount. Also disclosed are compositions of the invention for use in treating at least one of the aforesaid diseases, e.g. multiple sclerosis.

### BRIEF DESCRIPTION OF THE FIGURES

FIG. 1 is a box plot which demonstrates the effect of drug particle size on the content uniformity of a tablet comprising 5 mg (equivalent base weight) of the hemifumarate salt of 1-{4-[1-(4-cyclohexyl-3-trifluoromethyl-benzyloxyimino)-ethyl]-2-ethyl-benzyl}-azetidine-3-carboxylic acid.

FIG. 2 is a box plot which demonstrates the effect of drug particle size on the content uniformity of a tablet comprising 0.25 mg (equivalent base weight) of the hemifumarate salt of 1-{4-[1-(4-cyclohexyl-3-trifluoromethyl-benzyloxyimino)-ethyl]-2-ethyl-benzyl}-azetidine-3-carboxylic acid.

FIG. 3 is a line graph which compares the sum of degradation products of four different compositions, each comprising 0.25 mg (equivalent base weight) of the hemifumarate salt of 1-{4-[1-(4-cyclohexyl-3-trifluoromethyl-benzyloxyimino)-ethyl]-2-ethyl-benzyl}-azetidine-3-carboxylic acid mixed with a lubricant.

### DETAILED DESCRIPTION OF THE INVENTION

For the avoidance of doubt, it is hereby stated that the information disclosed earlier in this specification under the heading “Background to the Invention” is relevant to the invention and is to be read as part of the disclosure of the invention.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification (which term encompasses both the description and the claims) is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “treat” includes: (1) preventing or delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

“Effective amount” means an amount of an API or pharmaceutical composition sufficient to result in the desired therapeutic response. The therapeutic response can be any response that a user (e.g., a clinician or patient) will recognize as an effective response to the therapy.

The invention relates to oral pharmaceutical compositions comprising as an API an S1P receptor modulator or agonist, e.g. a compound of formula A1 or A2 as defined above. The disclosure describes solid phase dosage units, which may be a tablet or capsule, particularly a tablet. Exemplary compositions, e.g. tablets, contain 10 mg or less of the API (equivalent base weight), e.g. 7.5 mg or less of the API, for example 5 mg or less of the API. Some solid phase dosage units, e.g. tablets, contain 0.1 mg or more API (equivalent base weight), e.g. 0.2 mg or more of the API, for example 0.25 mg or more API. There are therefore included in the invention dosage units, e.g. tablets, which contain from 0.1 mg to 10 mg of the API (equivalent base weight), e.g. 0.2 mg to 7.5 mg of the API; particular dosage units, e.g. tablets, contain from 0.25 mg to 5 mg of the API (equivalent base weight), for example 2.5 mg to 5 mg of the API.

In one implementation of the invention, the dosage units (e.g. tablets) contain no more than 4 mg of the API (equivalent base weight), e.g. from 0.2 mg or 0.25 mg to 4 mg of the API. Some dosage units contain from 2 mg or 2.5 mg to 4 mg of the API (equivalent base weight).

In one embodiment, the dosage units (e.g. tablets) contain 4 mg of the API. In one embodiment, the dosage units (e.g. tablets) mentioned in this paragraph comprise, or consist of, a solid phase mixture consisting of API and non-basic excipients which comprise a cellulosic excipient, e.g. selected from microcrystalline cellulose, hypromellose, ethylcellulose and combinations thereof. In another embodiment, the dosage units (e.g. tablets) mentioned in this paragraph comprise, or consist of, a solid phase mixture consisting of API and non-basic excipients which comprise a controlled release material, e.g. a non-basic hydrogel-former such as, for example, hypromellose. The solid phase mixture may have at least one coating.

In one embodiment, the compound is of formula A1. It will be understood that this embodiment, like all embodiments mentioned herein, is applicable across the entire scope of the disclosure, including to all other embodiments disclosed herein, including those disclosed in the following paragraphs.

In one embodiment, R₅ is H.

In one embodiment, A is COOR₅, and in particular is COOH.

In one embodiment, Z is

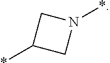

In one embodiment, A-Z is

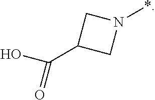

In one embodiment, Y is phenyl optionally substituted by 1, 2 or 3 radicals selected from halogen, NO₂, C₁₋₆alkyl, C₁₋₆alkoxy; halo-substituted C₁₋₆alkyl and halo-substituted C₁₋₆alkoxy, and in particular Y is phenyl substituted by a single said radical, e.g. by a single C₁₋₆ alkyl radical. The designation “C₁₋₆” means “having 1, 2, 3, 4, 5 or 6 carbon atoms” and an exemplary C₁₋₆ alkyl radical is ethyl.

In one embodiment, A—Z—C(R₄)(R₃)—Y— is

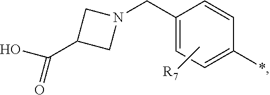

where R₇ is H, halogen, NO₂, C₁₋₆alkyl, C₁₋₆alkoxy; halo-substituted C₁₋₆alkyl and halo-substituted C₁₋₆alkoxy, and in particular is C₁₋₆alkyl e.g. ethyl.

In one embodiment, W is C₁, C₂ or C₃ alkylene, particularly methylene. Since every embodiment disclosed herein is applicable to the entire disclosure of the invention, it will be understood that in this case (where W is C₁, C₂ or C₃ alkylene, particularly methylene), the compound may be of the formula A1 and/or Y may be an optionally substituted phenyl group as previously described herein and in particular where A—Z—C(R₄)(R₃)—Y— may be

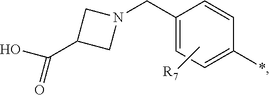

In one embodiment (for example applicable to the embodiments mentioned in the immediately preceding paragraph), R₁ is phenyl substituted by phenyl or by C₃-C₈ cycloalkyl, e.g. by cyclohexyl, wherein each phenyl and cyclohexyl are each independently optionally substituted by 1 or 2 substituents selected from halogen, C₁₋₆alkyl, C₁₋₆alkoxy and halo substituted —C₁₋₆alkyl or —C₁₋₆alkoxy. R₁ may therefore be phenyl substituted by C₃-C₈ cycloalkyl, each optionally substituted as just mentioned; in one sub-embodiment, phenyl of R₁ is substituted by a single substituent, e.g. fluoro or in particular trifluoromethyl, and cycloalkyl of R₁ is unsubstituted cyclohexyl. In an embodiment where the compound is of formula A1, phenyl is 1,4-substituted by W and cycloalkyl (e.g. unsubstituted cyclohexyl). The designation “C₃-C₈” means having 3, 4, 5, 6, 7 or 8 carbon atoms, e.g. 5 or 6 carbon atoms.

In one embodiment, the compound is of formula A1; W is C₁, C₂ or C₃ alkylene; Y is a phenyl group optionally substituted by 1, 2 or 3 radicals selected from halogen, NO₂, C₁₋₆alkyl, C₁₋₆alkoxy; halo-substituted C₁₋₆alkyl and halo-substituted C₁₋₆alkoxy; and R₁ is phenyl substituted by C₃-C₈ cycloalkyl, wherein phenyl and cyclohexyl are each independently optionally substituted by 1 or 2 substituents selected from halogen, C₁₋₆alkyl, C₁₋₆alkoxy and halo substituted —C₁₋₆alkyl or —C₁₋₆alkoxy.

### Pharmaceutical Compositions

The invention in its various aspects is in part predicated on a finding that an active compound as disclosed above, namely the hemifumarate salt of 1-{4-[1-(4-cyclohexyl-3-trifluoromethyl-benzyloxyimino)-ethyl]-2-ethyl-benzyl}-azetidine-3-carboxylic acid, has poor stability in certain settings and that stability of the compound in a pharmaceutical composition may be improved by adopting one, two or three of the following measures:
not exposing the active compound to a basic compound, e.g. excluding basic compounds from the composition (other than the active compound itself when in basic form).
having the active compound in the form of particles having a crystallinity of 80% or more.
having the active compound in the form of particles having an X90 diameter of at least 8 μm, e.g. at least 10 μm.

It will be appreciated that the hemifumarate salt of 1-{4-[1-(4-cyclohexyl-3-trifluoromethyl-benzyloxyimino)-ethyl]-2-ethyl-benzyl}-azetidine-3-carboxylic acid is a representative of the compounds of formula A1 or A2 as described above and that the just-mentioned measures may therefore be beneficially applied to any such compound as an active pharmaceutical ingredient (API) of a composition.

Advantageously, the composition is substantially dry, e.g. is made of substantially moisture-free constitutents, and is provided with a moisture-protective layer as a barrier to ingress of water and/or includes a desiccant.

In one implementation of the invention, the composition, for example in the form of a tablet, comprises a desiccant. In another implementation of the invention, the composition, for example in the form of a tablet, is provided with a moisture-protective layer. In a particular implementation, the composition, for example in the form of a tablet, comprises a desiccant and is provided with a moisture-protective layer.

It is advantageous to combine two or more of the above measures. For example embodiments of the invention lie in solid phase compositions, for examples capsules or tablets, particularly tablets, having one the following characteristics (1) to (6):
(1) the API is not exposed to basic compounds, e.g. basic compounds are excluded from the composition, and the API is in the form of particles having a crystallinity of 80% or more;
(2) the API is not exposed to basic compounds, e.g. basic compounds are excluded from the composition, and the API is in the form of particles having an X90 diameter of at least 8 μm e.g. at least 10 μm;
(3) the API is in the form of particles having an X90 diameter of at least 8 μm e.g. at least 10 μm and a crystallinity of 80% or more;
(4) the API is not exposed to basic compounds, e.g. basic compounds are excluded from the composition, and the API is in the form of particles having an X90 diameter of at least 8 μm e.g. at least 10 μm and a crystallinity of 80% or more;
(5) any of (1), (2), (3) and (4) in combination with inclusion of a desiccant in the composition;
(6) any of (1), (2), (3), (4) and (5) in combination with provision of a moisture-protective layer or coating.

In particular, the above-mentioned APIs have poor stability in admixture with one or more of the following compounds; sodium starch glycolate (sold under the trade mark Primojel®), sodium lauryl sulfate, magnesium stearate, calcium stearate, calcium carbonate, calcium sulfate, sodium stearyl fumarate and sodium bicarbonate. The above-mentioned APIs also have poor stability in admixture with gelatin.

Not exposing the active compound to a basic compound may be achieved by not having the active compound in admixture with a basic compound. In an embodiment, therefore, the API is in a mixture of materials which is free of basic compounds. Conveniently, therefore, the composition is free of basic compounds. However, the invention includes, for example, the provision of two-or multi-part compositions of which one part incorporates a compound of formula A1 or A2 but is free of any basic compound and a second part does not incorporate a compound of formula A1 or A2 but does incorporate a basic compound. It will be understood that the expression “free of any basic compound” and similar expressions do not mean that no base may be present in the composition but does allow very low amounts of base to be present, such an amount therefore being at a concentration which does not for practical purposes promote degradation of the API. For example, a basic compound may be an impurity which it is not possible or practicable to remove entirely or to an undetectable level.

In embodiments, moiety A is not in the form of a salt but R₅ is present as H or an ester-forming group. R₅ particularly is present as H (i.e. moiety A is in its acid form and not as a salt).

In one embodiment the pharmaceutical composition comprises a solid phase mixture, which may be in the form of a tablet, and which consists of a compound of formula A1 or A2 in admixture with one or more non-basic compounds. The one or more non-basic compounds may comprise or consist of non-basic excipients, e.g. selected from: binders, disintegrants, glidants, lubricants, fillers, diluents, controlled release agents and sorbents. The composition, e.g. tablet, may have one or more coating layers and/or may have an basic component separated from said solid phase mixture by a barrier. Said solid phase mixture may comprise one or more non-basic APIs in addition to one or more APIs of formula A1 or A2. The composition, e.g. tablet, may have a coating layer which is a moisture barrier, for example as sold under the trade mark Opadry® amb. A moisture barrier film composition is described in WO1996/001874, included herein by reference for all purposes, and comprises polyvinyl alcohol, soya lecithin, and optionally a flow aid, a colorant, and/or a suspending agent. Conveniently, the composition includes a desiccant, e.g. colloidal silica.

In one embodiment the pharmaceutical composition comprises a solid phase mixture, which may be in the form of a tablet, and which consists of a compound of formula A1 or A2 in admixture with excipients selected from lactose (e.g. as lactose monohydrate); microcrystalline cellulose; non-basic polymers e.g. homopolymers of cross-linked N-vinyl-2-pyrrolidone (crospovidone), hypromellose (hydroxypropylmethyl cellulose), and ethyl cellulose; waxes; colloidal silicon dioxide; stearic acid; hydrogenated vegetable oil; mineral oil; polyethylene glycol (e.g. polyethylene glycol 4000-6000); gyceryl palmitostearate; and glyceryl behenate. In such a formulation, lactose is considered to act as a filler, microcrystalline cellulose as a binder, crospovidone as a disintegrant, hypromellose and ethyl cellulose as a controlled release agent, colloidal silicon dioxide as a glidant and the remaining materials as lubricants; in embodiments, only a single such lubricant is included in the composition, e.g. glyceryl behenate. Colloidal silicon dioxide acts also as a desiccant. Hydrogenated vegetable oils may act as controlled release agents.

In one embodiment, the pharmaceutical composition comprises, or consists of, a solid phase mixture consisting of API and non-basic excipients which comprise a cellulosic excipient, e.g. selected from microcrystalline cellulose, hypromellose, ethylcellulose and combinations thereof. In another embodiment, the pharmaceutical composition comprises, or consists of, a solid phase mixture consisting of API and non-basic excipients which comprise a controlled release material, e.g. a non-basic hydrogel-former such as, for example, hypromellose. The solid phase mixture may have at least one coating.

An embodiment of the invention therefore comprises a solid phase mixture, which may be in the form of a tablet, and which consists of a compound of formula A1 or A2 in admixture with: lactose (e.g. as lactose monohydrate); microcrystalline cellulose; a polymer selected from homopolymers of cross-linked N-vinyl-2-pyrrolidone (crospovidone), hypromelloses, and ethyl cellulose; waxes, colloidal silicon dioxide; and a lubricant selected from stearic acid, hydrogenated vegetable oil, mineral oil, polyethylene glycol (e.g. polyethylene glycol 4000-6000), gyceryl palmitostearate and glyceryl behenate, and combinations of the aforesaid lubricant compounds. For example, such an embodiment may comprise a solid phase mixture, which may be in the form of a tablet, and which consists of a compound of formula A1 or A2 in admixture with: lactose (e.g. as lactose monohydrate); microcrystalline cellulose; a polymer selected from homopolymers of cross-linked N-vinyl-2-pyrrolidone (crospovidone) and hypromelloses; colloidal silicon dioxide; and a lubricant selected from hydrogenated vegetable oil, mineral oil, polyethylene glycol (e.g. polyethylene glycol 4000-6000), gyceryl palmitostearate and glyceryl behenate, and combinations of the aforesaid lubricant compounds. In embodiments, a single lubricant is present in the solid phase mixture, particularly glyceryl behenate.

### Particle Size

It will be recalled that an aspect of the invention is in part predicated on a finding that the stability in a solid phase composition, particularly a tablet, of an active compound as disclosed above, namely the hemifumarate salt of 1-{4-[1-(4-cyclohexyl-3-trifluoromethyl-benzyloxyimino)-ethyl]-2-ethyl-benzyl}-azetidine-3-carboxylic acid, may be improved by the active compound being in a relatively large particle size but, in most instances, a particle size not so large that the composition fails to comply with the the USP, EP and JP harmonised content uniformity requirement, e.g. as in force on 1 Jan. 2011.

It will be appreciated that the hemifumarate salt of 1-{4-[1-(4-cyclohexyl-3-trifluoromethyl-benzyloxyimino)-ethyl]-2-ethyl-benzyl}-azetidine-3-carboxylic acid is a representative of the compounds of formula A1 or A2 as described above and that the just-mentioned measures relating to particle size may therefore be beneficially applied to any such compound as an active pharmaceutical ingredient (API) of a composition. The invention therefore provides a solid phase formulation, particularly a tablet, comprising a compound of formula A1 or A2 in a relatively large particle size, e.g. an X90 diameter of at least 8 μm and often of 10 μm or more, for example 20 μm or 25 μm or more, optionally of 100 μm or more and further optionally of 150 μm or more. The particle size (X90 diameter) may be up to 300 μm, e.g. up to 250 μm, and optionally up to 200 μm (e.g. up to 195 μm). In some embodiments, the X90 diameter is of from 10 μm to 300 μm, e.g. 10 μm to 250 μm or 10 μm to 200 μm. Also included are embodiments in which the particle size (X90 diameter) is from 25 μm to 300 μm, e.g. 25 μm to 250 μm or 25 μm to 200 μm. Further included are embodiments in which the X90 diameter is from 100 μm to 300 μm, e.g. 100 μm to 250 μm or 100 μm to 200 μm. In a particular embodiment, the X90 particle diameter is at least 10 μm and is less than 191 μm, e.g. less than 180 μm, less than 170 μm, less than 160 μm, less than 150 μm, less than 140 μm or less than 130 μm, e.g. no more than 125 μm, such as no more than 121 μm; in a sub-embodiment, the X90 particle diameter is at least 25 μm and is less than 191um, e.g. less than 180um, less than 170 μm, less than 160 μm, less than 150 μm, less than 140 μm or less than 130 μm, e.g. no more than 125 μm, such as no more than 121 μm. In some instances the X90 particle diameter is at least 10 μm or 25 μm but no more than 100 μm, e.g. is no more than 90 μm, no more than 80 μm, no more than 70 μm, no more than 60 μm, no more than 50 μm or no more than 40 μm, for example is no more than 30 μm. In other embodiments, the X90 particle diameter is from 121 μm to 191 μm. In another embodiment, the X90 particle diameter is from 11 μm to 29 μm.

An aspect of the invention is in part predicated on a finding that, in the case of an API of formula A1 or A2, API particles of the sizes mentioned herein of at least 8um may be formulated into tablets which have a tablet content uniformity which fulfills the requirement of the USP, EP and JP harmonised content uniformity requirement, in particular as in force on 1 Jan. 2011. The pharmaceutical compositions of the present invention may fulfil, and desirably do fulfill, the USP, EP and JP harmonised content uniformity requirement (refer to chapter 2.9.40. Uniformity of dosage unit in the EP). A maximum particle size which enables the requirement to be met cannot be stipulated because the maximum possible particles size varies with the drug content of the dosage unit. For example, in the case of a tablet containing 0.25 mg of an API (equivalent base weight), API particles having an X90 diameter of 121 μm complied with the harmonised content uniformity requirement whereas API particles having an X90 diameter of 191 μm failed to comply. In contrast, in the case of a tablet containing 5 mg (equivalent base weight), of an API, API particles having an X90 diameter of up to 191 μm complied with the harmonised content uniformity requirement. A maximum particle size which enables any particular tablet composition to meet the harmonised content uniformity requirement may be determined empirically.

The particle size distribution (by volume) may be measured using a laser diffraction sizing instrument, for example the Sympatec Helos device (available from Sympactec GmbH, Germany) using the Cuvette dispersion device. The X₉₀ diameter is the spherical diameter corresponding to the X₉₀ volume.

To make the measurement, a stock dispersion may be prepared by mixing the drug substance with a dispersing aid (e.g Octastat 5000 (Octel corp)) using a vortex until a smooth and homogeneous paste is formed. The paste may then be diluted and mixed to a final volume of 3 to 6 ml using white spirit. The optical concentration of the final solution should remain below 5%. The percent values are calculated from the mean cumulative volume size curve by the software of the Sympatec instrument. X90 mean 90% of the particle size population is below the specified value in volume.

Additional confirmatory information on particle size may be obtained using SEM (scanning electron microscopy).

Content uniformity testing is used to test the active content within individual units post-manufacturing (such as the content of active agents within individual tablets after compression).

Content uniformity is influenced by the particle size of the drug substances. Median particle size is denoted by Xₘ, where m is a percentage of the particle size distribution.

The invention includes the following embodiments (optionally in combination with the features of other embodiments disclosed herein):
1) a solid phase pharmaceutical composition in unit dosage form which comprises one or more pharmaceutically acceptable excipients, in particular non-basic excipients, and an API as described herein and which complies with the US Pharmacopeia, European Pharmacopeia and Japanese Pharmacopeia harmonised content uniformity requirements as in force on 1 Jan. 2011;
2) a tablet which comprises one or more pharmaceutically acceptable excipients, in particular non-basic excipients, and an API as described herein in an amount of from 4 mg to 6 mg API (equivalent base weight), the API being as particles have an X90 diameter of at least 100 μm but no more than 300 μm, e.g. from 100 μm to 250 μm, from 100 μm to 200 μm, from 100 μm to 190 μm, from 100 μm to 180 μm, from 100 μm to 170 μm, from 100 μm to 160 μm or from 100 μm to 150 μm;
3) a tablet which comprises one or more pharmaceutically acceptable excipients, in particular non-basic excipients, and an API as described herein in an amount of from from 0.2 mg to 1 mg API (equivalent base weight), the API being as particles have an X90diameter of at least 10 μm but no more than 100 μm, e.g. 10 μm to 90 μm, 10 μm to 80 μm, 10 μm to 70 μm, 10 μm to 60 μm, 10 μm to 50 μm, 10 μm to 40 μm or 10 μm to 30 μm.

An aspect of the invention resides in compounds of formula A1 or A2 when in particulate form having an X90 diameter as disclosed herein, as well as products or compositions of matter comprising such particulate compounds.

### Crystallinity

The invention is in part also predicated on a finding that the stability in a solid phase formulation, particularly a tablet, of an active compound as disclosed above, namely the hemifumarate salt of 1-{4-[1-(4-cyclohexyl-3-trifluoromethyl-benzyloxyimino)-ethyl]-2-ethyl-benzyl}-azetidine-3-carboxylic acid, may be enhanced by the active compound being in particles of relatively high crystallinity, e.g. a crystallinity of 80% or more, for example 85% or more and optionally of 90% or more.

It will be appreciated that the above salt of 1-{4-[1-(4-cyclohexyl-3-trifluoromethyl-benzyloxyimino)-ethyl]-2-ethyl-benzyl}-azetidine-3-carboxylic acid is a representative of the compounds of formula A1 or A2 as described above and that the just-mentioned measures may therefore be beneficially applied to any such compound as an active pharmaceutical ingredient (API) of a composition. The invention therefore provides a solid phase formulation, particularly a tablet, comprising a compound of formula A1 or A2 in a crystallinity of 80% or more, for example 85% or more and optionally of 90% or more.

An aspect of the invention resides in compounds of formula A1 or A2 when having a crystallinity of 80% or more, for example 85% or more and optionally of 90% or more, as well as products or compositions of matter comprising compounds having such crystallinity.

Conveniently, a crystallinity of 80% or more may be attained by a compound of formula A1 or A2 being as particles having an X90 diameter of 10 μm or more and particularly 11 μm or more, for example 20 μm or 25 μm or more, optionally of 100 μm or more and further optionally of 150 μm or more. The X90 diameter may be as previously described herein under the heading “Particle Size”.

Crystallinity may be measured using any suitable means, for example using X-ray powder diffraction (XRPD), for example using a Bruker D8 device.

It will be appreciated that solid phase compositions, e.g. tablets, may advantageously combine the features of particle size and/or crystallinity disclosed herein with the feature of not exposing the API to a basic compound.

### Methods for Making the Pharmaceutical Compositions

Compositions of the invention may suitably be made by combining the components as dry powders, for example tablets may be made by dry granulating the components of the tablet mix and optionally applying a film coating, for example a moisture barrier film, to the compressed tablet.

API particles can be prepared by suitable milling techniques, e.g. those well known in the art such as, for example, wet-jet milling, pin-milling, and wet-ball milling

Where the API particles are derived from coarse API particle crystals, the coarse crystals may be obtained using any suitable methodolgy. For example any of the methodologies set out in WO2010/071794, WO2010/080455 or WO2010/080409.

Of interest in the invention are the following numbered paragraphs.

1. A solid phase pharmaceutical composition comprising one or more pharmaceutically acceptable excipients and an active pharmaceutical ingredient (“API”) which is a compound of formula A1 or A2 or a pharmacologically acceptable salt, solvate or hydrate thereof, wherein the API is not exposed to a basic compound:

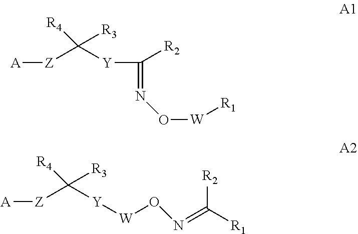

wherein
A is COOR₅, OPO(OR₅)₂, PO(OR₅)₂, SO₂OR₅, POR₅OR₅ or 1H-tetrazol-5-yl, R₅ being H or an ester-forming group;
W is a bond, C₁₋₃alkylene or C₂₋₃alkenylene;
Y is C₆₋₁₀aryl or C₃₋₉heteroaryl, optionally substituted by 1 to 3 radicals selected from halogen, NO₂, C₁₋₆alkyl, C₁₋₆alkoxy; halo-substituted C₁₋₆alkyl and halo-substituted C₁₋₆alkoxy;

Z is chosen from:

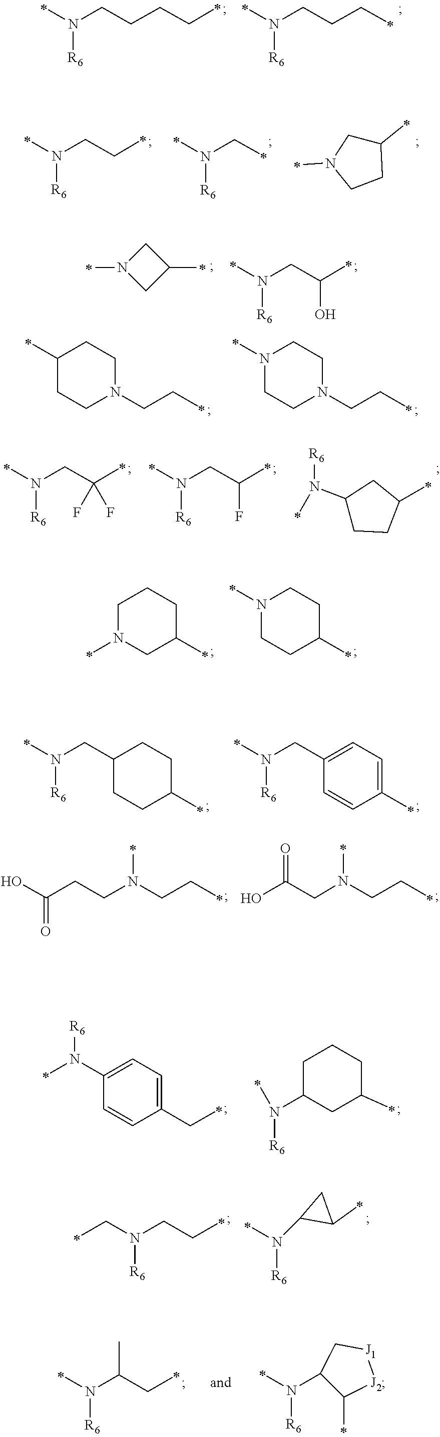

wherein the asterisks of Z indicate the point of attachment between —C(R₃)(R₄)— and A of Formula Ia or Ib, respectively; R₆ is chosen from hydrogen and C₁₋₆alkyl; and J₁ and J₂ are independently methylene or a heteroatom chosen from S, O and NR₅; wherein R₅ is chosen from hydrogen and C₁₋₆alkyl; and any alkylene of Z can be further substituted by one to three radicals chosen from halo, hydroxy, C₁₋₆alkyl; or R₆ can be attached to a carbon atom of Y to form a 5-7 member ring;
R₁ is C₆₋₁₀aryl or C₃₋₉heteroaryl, optionally substituted by C₁₋₆alkyl, C₆₋₁₀aryl, C₆₋₁₀arylC₁₋₄alkyl, C₃₋₉heteroaryl, C₃₋₉heteroarylC₁₋₄alkyl, C₃₋₈cycloalkyl, C₃₋₈cycloalkylC₁₋₄alkyl, C₃₋₈heterocycloalkyl or C₃₋₈heterocycloalkylC₁₋₄alkyl; wherein any aryl, heteroaryl, cycloalkyl or heterocycloalkyl of R₁ may be substituted by 1 to 5 groups selected from halogen, C₁₋₆alkyl, C₁₋₆alkoxy and halo substituted—C₁₋₆alkyl or —C₁₋₆alkoxy;
R₂ is H, C₁₋₆alkyl, halo substituted C₁₋₆alkyl, C₂₋₆alkenyl or C₂₋₆alkynyl; and
each of R₃ or R₄, independently, is H, halogen, OH, C₁₋₆alkyl, C₁₋₆alkoxy or halo substituted C₁₋₆alkyl or C₁₋₆alkoxy.

2. A composition of numbered paragraph 1 wherein A is COOH.

3. A composition of numbered paragraph 1 or 2 wherein the compound is of formula A1.

4. A composition of numbered paragraph 3 wherein:
W is C₁, C₂ or C₃ alkylene;
Y is a phenyl group optionally substituted by 1, 2 or 3 radicals selected from halogen, NO₂, C₁₋₆alkyl, C₁₋₆alkoxy; halo-substituted C₁₋₆alkyl and halo-substituted C₁₋₆alkoxy; and
R₁ is phenyl substituted by C₃-C₈ cycloalkyl, wherein phenyl and cyclohexyl are each independently optionally substituted by 1 or 2 substituents selected from halogen, C₁₋₆alkyl, C₁₋₆alkoxy and halo substituted—C₁₋₆alkyl or —C₁₋₆alkoxy.

5. A composition of numbered paragraph 1 wherein the API is 1-{4-[1-(4-cyclohexyl-3-trifluoromethyl-benzyloxyimino)-ethyl]-2-ethyl-benzyl}-azetidine-3-carboxylic acid or a pharmaceutically acceptable salt.

6. A composition of numbered paragraph 1 wherein the API is 1-{4-[1-(4-cyclohexyl-3-trifluoromethyl-benzyloxyimino)-ethyl]-2-ethyl-benzyl}-azetidine-3-carboxylic acid or a hemifumarate salt thereof.

7. A composition of any preceding numbered paragraph wherein the API is in a mixture of materials which is free of basic compounds.

8. A composition of any preceding numbered paragraph which is a tablet.

9. A composition of numbered paragraph 8 wherein the tablet is coated with a moisture barrier.

10. A composition of numbered paragraph 8 or 9 wherein the tablet comprises a solid phase mixture which consists of the API in admixture with: lactose; microcrystalline cellulose; a polymer selected from homopolymers of cross-linked N-vinyl-2-pyrrolidone (crospovidone) and hypromelloses; colloidal silicon dioxide; and a lubricant selected from hydrogenated vegetable oil, mineral oil, polyethylene glycol, gyceryl palmitostearate and glyceryl behenate.

11. A solid phase pharmaceutical composition comprising one or more pharmaceutically acceptable excipients and an active pharmaceutical ingredient (“API”) selected from the compounds as defined in any of numbered paragraphs 1 to 6, wherein the API is in the form of particles having an X90 diameter of at least 8 μm.

12. A composition of numbered paragraph 11 wherein the particles have an X90 diameter of from 10 μm to 300 μm.

13. A composition of numbered paragraph 12 wherein the particles have an X90 diameter of at least 10 μm but no more than 100 μm.

14. A composition of numbered paragraph 12 wherein the particles have an X90 diameter of at least 100 μm but no more than 250 μm.

15. A composition of any of numbered paragraphs 11 to 14 which is in unit dosage form and complies with the US Pharmacopeia, European Pharmacopeia and Japanese Pharmacopeia harmonised content uniformity requirements as in force on 1 Jan. 2011.

16. A composition of numbered paragraph 15 which is in the form of a tablet containing from 4 mg to 6 mg API (equivalent base weight) and particles have an X90 diameter of at least 100 μm but no more than 200 μm.

17. A composition of numbered paragraph 15 which is in the form of a tablet containing from 0.2 mg to 1 mg API (equivalent base weight) and particles have an X90 diameter of at least 10 μm but no more than 50 μm.

18. A composition of any of numbered paragraphs 11 to 17 which further includes the features of any one of, or any combination permitted by dependency of, of numbered paragraphs 1 to 10.

19. A solid phase pharmaceutical composition comprising one or more pharmaceutically acceptable excipients and an active pharmaceutical ingredient (“API”) selected from the compounds as defined in any of numbered paragraphs 1 to 6, wherein the API has a crystallinity of 80% or more.

20. A composition of numbered paragraph 19 wherein the API has a crystallinity of 85% or more.

21. A composition of numbered paragraph 19 or 20 which is a tablet.

22. A composition of any of numbered paragraphs 18 to 21 which further includes the features of any one of, or any combination permitted by dependency of, of numbered paragraphs 1 to 10 and 11 to 17.

23. A tablet comprising a compressed mixture consisting of 1-{4-[1-(4-cyclohexyl-3-trifluoromethyl-benzyloxyimino)-ethyl]-2-ethyl-benzyl}-azetidine-3-carboxylic acid or a pharmaceutically acceptable salt thereof and one or more non-basic excipients, the 1-{4-[1-(4-cyclohexyl-3-trifluoromethyl-benzyloxyimino)-ethyl]-2-ethyl-benzyl}-azetidine-3-carboxylic acid or pharmaceutically acceptable salt being in the form of particles having an X90 diameter of from 10 μm to 200 μm.

24. A tablet of numbered paragraph 23, wherein the pharamaceutically acceptable salt is a hemifumarate salt.

25. A tablet of numbered paragraph 23 or 24 wherein said particles are at least 80% crystalline.

26. A tablet of numbered paragraph 23, 24 or 25 wherein the compressed mixture includes a desiccant and is coated with a moisture barrier.

Specific tablet embodiments of the formulations of the present invention wherein the active agent, 1-{4-[1-(4-cyclohexyl-3-trifluoromethyl-benzyloxyimino)-ethyl]-2-ethyl-benzyl}-azetidine-3-carboxylic acid, is present in amounts of 0.25 mg, 0.5 mg, and 1.0 mg, respectively, are described in the chart below.

| Dosage strenght | 0.25 mg | | 0.5 mg | | 1.0 mg | |
|---|---|---|---|---|---|---|
| Per FCT | mg | % | mg | % | mg | % |
| BAF312 | 0.278 | 0.310 | 0.556 | 0.621 | 1.112 | 1.241 |
| Glyceryl behenate | 4.25 | 4.743 | 4.25 | 4.743 | 4.25 | 4.743 |
| Lactose Spray dried | 62.197 | 69.416 | 61.919 | 69.106 | 61.363 | 68.485 |
| Cellulose | 12.75 | 14.230 | 12.75 | 14.230 | 12.75 | 14.230 |
| Polyvinyl Polypyrolidone XL | 5.1 | 5.692 | 5.1 | 5.692 | 5.1 | 5.692 |
| Aerosil | 0.425 | 0.474 | 0.425 | 0.474 | 0.425 | 0.474 |
| Total TAB | 85 | 94.866 | 85 | 94.866 | 85 | 94.866 |
| Opadry AMB ® | 4.6 | 5.134 | 4.6 | 5.134 | 4.6 | 5.134 |
| total FCT | 89.6 | 100.000 | 89.6 | 100.000 | 89.6 | 100.000 |

Another specific tablet embodiment of the formulations of the present invention wherein the active agent, 1-{4-[1-(4-cyclohexyl-3-trifluoromethyl-benzyloxyimino)-ethyl]-2-ethyl-benzyl}-azetidine-3-carboxylic acid, is present in an amount of 2.0 mg, is described in the chart below.

| Dosage strenght | 2.0 mg | |
|---|---|---|
| Per FCT | mg | % |
| BAF312 | 2.224 | 2.482 |
| Glyceryl behenate | 4.25 | 4.743 |
| Lactose Spray dried | 60.251 | 67.244 |
| Cellulose | 12.75 | 14.230 |
| Polyvinyl Polypyrolidone XL | 5.1 | 5.692 |
| Aerosil | 0.425 | 0.474 |
| Total TAB | 85 | 94.866 |
| Opadry AMB ® | 4.6 | 5.134 |
| total FCT | 89.6 | 100.000 |

### EXAMPLES

### Example 1

The following Example illustrates the stability of the hemifumarate salt of 1-{4-[1-(4-cyclohexyl-3-trifluoromethyl-benzyloxyimino)-ethyl]-2-ethyl-benzyl}-azetidine-3-carboxylic acid in the presence of various compounds. Mixtures of this API with various compounds were exposed in a sealed vial to conditions of 50° C. temperature and 75% relative humidity for 4 weeks. After 4 weeks, the API degradation and sum of degradation products were assayed. The results are displayed in Table 1.

Assay method: HPLC using agilent 11000 and a Phenomenex Gemini C18 column. UV detection at 260 nm was used. The mobile phase used was a gradient water-acetonitrile containing 0.2% (v/v) formic acid and 10% (v/v) Triethylamine.

The hemifumarate salt of 1-{4-[1-(4-cyclohexyl-3-trifluoromethyl-benzyloxyimino)-ethyl]-2-ethyl-benzyl}-azetidine-3-carboxylic acid is described as “NVS-A” in Table 1.

TABLE 1

| | Sum of | |
|---|---|---|
| | unknown | Assay |
| Mixture | peaks (%)* | (%)* |
| NVS-A alone | 1.86 | 100.2 |
| NVS-A + Lactose (1:1) | 1.74 | 96.5 |
| NVS-A + Maize Starch (1:1) | 1.82 | 99.4 |
| NVS-A + Microcrystalline Cellulose | 1.80 | 99.0 |
| (1:1) | | |
| NVS-A + Mannitol (1:1) | 1.80 | 99.2 |
| NVS-A + HPMC (1:1) | 2.45 | 97.7 |
| NVS-A + PVP K30 (1:1) | 2.07 | 99.6 |
| NVS-A + Ac-Di-Sol (1:1) | 1.89 | 99.7 |
| NVS-A + Primojel (1:1) | 4.08 | 100.9 |
| NVS-A + Crospovidone (1:1) | 1.89 | 99.7 |
| NVS-A + Mgstearate (1:0.85) | 0.31 | 98.2 |
| NVS-A + Mg Stearate (1:0.85)* | 3.68 | 90.0 |
| NVS-A + Sodium LaurylSulfate (SLS) + | 8.54 | 88.3 |
| Microcrystalline Cellulose (2:1:1) | | |
| NVS-A + Gelatin (1:1) | 3.86 | 96.1 |
| NVS-A + Aerosil + Microcrystalline | 1.89 | 99.9 |
| Cellulose (2:1:1) | | |
| NVS-A + Magnesium Stearate (Mg St) + | 6.13 | 89.2 |
| Microcrystalline Cellulose (2:1:1) | | |
| NVS-A + Magnesium stearate + | 10.53 | 80.7 |
| Microcrystalline Cellulose (2:1:1 | | |
| NVS-A + Magnesium stearate + lactose | 9.31 | 82.2 |
| (2:1:1) | | |
| NVS-A + Calcium stearate + | 4.55 | 89.8 |
| Microcrystalline Cellulose (2:1:1) | | |
| NVS-A + sodium stearyl fumarate + | 7.52 | 83.1 |
| Microcrystalline Cellulose (2:1:1) | | |
| NVS-A + Sodium bicarbonate + | 8.48 | 61.8 |
| Microcrystalline Cellulose (2:1:1) | | |
| NVS-A + Tween 80 + Microcrystalline | 2.07 | 100.1 |
| Cellulose (2:1:1) | | |

*at 50° C./75% RH opened vial/4 weeks

### Example 2

The following Example demonstrates the detrimental effect of magnesium stearate on the hemifumarate salt of 1-{4-[1-(4-cyclohexyl-3-trifluoromethyl-benzyloxyimino)-ethyl]-2-ethyl-benzyl}-azetidine-3-carboxylic acid (NVS-A).

A film coated tablet containing NVS-A was developed comprising micronised drug substance, lactose monohydrate, microcrystalline cellulose, crospovidone, colloidal silicon dioxide, and magnesium stearate. A moisture protective Opadry® AMB film coating was applied. Tablet strengths from 0.25 mg to 5 mg (equivalent free base weight) were produced as shown in Table 2 below.

TABLE 2
Composition of the immediate release 0.25 and 5 mg tablet
containing magnesium stearate as lubricant.

| | | | | |
|---|---|---|---|---|
| | Amount | Amount | | |
| | (mg) per | (mg) per | | Reference |
| | 0.25 mg | 5 mg | | to |
| Ingredient | tablet | tablet | Function | standards |

Tablet core

| | | | | |
|---|---|---|---|---|
| NVS-A | 0.278¹ | 5.560² | Drug | Novartis |
| | | | substance | monograph |
| Lactose | 63.897 | 58.615 | Diluent | Ph. Eur./NF |
| monohydrate | | | | |
| Microcrystalline | 17.000 | 17.000 | Diluent | Ph. Eur./NF |
| cellulose/ | | | | |
| Cellulose | | | | |
| microcrystalline | | | | |
| Crospovidone | 2.550 | 2.550 | Disintegrant | Ph. Eur./NF |
| Silica, colloidal | 0.425 | 0.425 | Gliding | Ph. Eur./NF |
| anhydrous/ | | | agent | |
| Colloidal silicon | | | | |
| dioxide | | | | |
| Magnesium | 0.850 | 0.850 | Lubricant | Ph. Eur./NF |
| stearate | | | | |
| Core tablet | 85.000 | 85.000 | | |
| weight | | | | |

Coating

| | | | | |
|---|---|---|---|---|
| Coating premix | 3.400 | 3.400 | Coating | |
| Opadry AMB | | | agent | |
| white** | | | | |
| Water, purified* | 13.600 | 13.600 | Solvent | Ph.Eur./ |
| Total film-coated | 88.400 | 88.400 | | USP |
| tablet weight | | | | |

Tablet core

| Coating premix ingredient | Reference to standards |
|---|---|
| Polyvinyl alcohol-part | Ph.Eur./USP |
| hydrolised | |
| Titanium dioxide | Ph.Eur./USP |
| Talc | Ph.Eur./USP |
| Lecithin (soya) | NF |
| Xanthan gum | Ph.Eur./NF |

¹Corresponds to 0.25 mg (e.g. 0.294% w/w) NVS-A base respectively
²Corresponds to 5 mg (e.g 5.88% w/w) NVS-A base respectively
*Removed during processing
**The qualitative composition of the coating premix is as follows:

The tablets were only stable for prolonged periods at 2-8° C. At 25° C., the 0.25 mg strength tablet was stable for only 6 months and the 5 mg tablet was stable for only 12 months. Beyond 6 months and 12 months at 25° C. respectively for the 0.25 and 5 mg tablet, the stability data did not fulfill the stability guidance from the International Conference on Harmonisation of Technical Requirements for Registration of Pharmaceuticals for Human Use (ICH).

### Example 3

The effect of drug particle size on the content uniformity of a tablet comprising 5 mg (equivalent free base weight) of the hemifumarate salt of 1-{4-[1-(4-cyclohexyl-3-trifluoromethyl-benzyloxyimino)-ethyl]-2-ethyl-benzyl}-azetidine-3-carboxylic acid (NVS-A) was studied.

The X₉₀ particle size distribution (by volume) was measured using a laser diffraction sizing instrument [the Sympatec Helos device (available from Sympactec GmbH, Germany) using the Cuvette dispersion device. To make the measurement, a stock dispersion was prepared by mixing the drug substance with a dispersing aid (Octastat 5000 (Octel corp)) using a vortex until a smooth and homogeneous paste was formed. The paste was then diluted and mixed to a final volume of 3 to 6 ml using white spirit. The optical concentration of the final solution were kept below 5%. The percentage values were calculated from the mean cumulative volume size curve by the software of the Sympatec instrument. Additional confirmatory information on particle size was obtained using SEM (scanning electron microscopy).

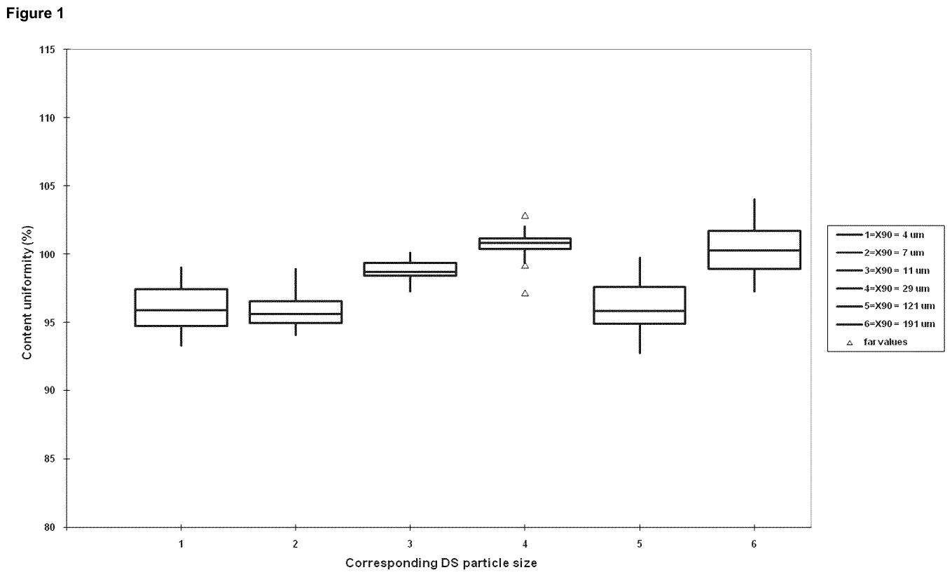

The finer API material (X₉₀<10 μm (4 and 7 μm) was obtained by micronisation using a jet mill [Hosokawa Alpine AFG100] using a pressure in the range from 2 to 5 bars. The material with X₉₀ at 11, 29 μm and 121 μm was obtained by milling in this case with a pin mill with the relative tin speed being adjusted from 115, to 75 to 40 m/sec respectively. Finally, the coarser material with X₉₀ μm of 191 μm was obtained by sieving on a 457 μm sieve. The results are presented in FIG. 1.

The drug substance particle sizes considered showed reduced impact on the mean content uniformity value and its variability for the 5 mg tablet:
where X90=191 μm, the content uniformity ranged from about 97.3% to about 104.0%.
where X90=121 μm, the content uniformity ranged from about 92.8% to about 99.7%.
where X90=29 μm, the content uniformity ranged from about 97.2% to about 102.9%.
where X90=11 μm, the content uniformity ranged from about 97.3% to 100.1%.
where X90=7 μm, the content uniformity ranged from about 94.1% to 98.9%.
where X90=4 μm, the content uniformity ranged from about 93.3% to 99.1%.

This study demonstrated that despite the low tablet strength, micronised drug substance (where X90=4 μm to 7 μm) was not essential to fulfil the USP, EP and JP harmonised content uniformity requirement. This requirement could be achieved when using milled drug substance (where X90=11 μm to 29 μm) and also unexpectedly using the coarse drug substance (where X90=121 μm to 191 μm).

### Example 4

The effect of drug particle size on the content uniformity of a tablet comprising 0.25 mg (equivalent free base weight) of the hemifumarate salt of 1-{4-[1-(4-cyclohexyl-3-trifluoromethylbenzyloxyimino)-ethyl]-2-ethyl-benzyl}-azetidine-3-carboxylic acid (NVS-A) was studied. API drug particles were obtained and measured as in Example 3.

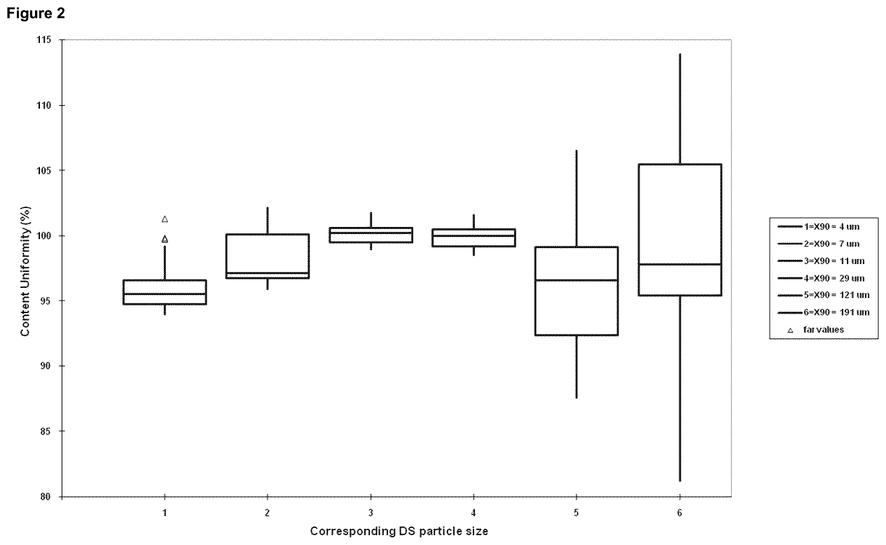

The results are presented in FIG. 2.

Coarse drug substance with an X90 diameter of 121 μm and 191 μm led to a significant increase in content uniformity variability:
where X90 =191 μm, the content uniformity ranged from about 81.2% to about 113.9%.
where X90 =121 μm, the content uniformity ranged from about 87.6% to about 106.5%.

Milled drug substance with an X90 of 11 μm or 29 μm led to a better centered mean content uniformity and a slightly lower content variability than the one observed with the micronised drug substance (X90=4 and 7 μm):
where X90=29 μm, the content uniformity ranged from about 98.5% to about 101.6%.
where X90=11 μm, the content uniformity ranged from about 98.9% to about 101.8%.
where X90=7 μm, the content uniformity ranged from about 95.9% to about 102.2%.
where X90=4 μm, the content uniformity ranged from about 94.0% to about 101.3%.

This example demonstrates that despite the low tablet strength, micronised drug substance (where X90=4 μm to 7 μm) is not essential to fulfil the USP, EP and JP harmonised content uniformity requirement. This requirement can unexpectedly be achieved when using milled drug substance (where X90=11 μm to 29 μm). The upper limit that achieved the USP, EP and JP harmonised content uniformity requirement was a 0.25 mg tablet with a particle size characterised by a X90=121 μm.

### Example 5

The following example compares the sum of degradation products of four different compositions, each comprising 0.25 mg (equivalent free base weight) of the hemifumarate salt of 1-{4-[1-(4-cyclohexyl-3-trifluoromethyl-benzyloxyimino)-ethyl]-2-ethyl-benzyl}-azetidine-3-carboxylic acid (NVS-A) mixed with a lubricant. API drug particles were obtained and measured as in Example 3. The lubricants and the particle sizes of each composition are as follows. The table below summarises the difference in composition between the tablets made with magnesium stearate and glyceryl behenate as lubricant. It should be noted that the tablet made with glyceryl behenate and with the different drug substance particle size were not coated with the moisture protective film coated opadry AMB in order to better demonstrate the stability benefit. API drug particles were obtained and measured as in

TABLE 3
Composition of the immediate release 0.25 mg tablet
(equivalent free base weight) containing magnesium
stearate and glyceryl behenate as lubricant.

| | | | | |
|---|---|---|---|---|
| | Amount | Amount | | |
| | (mg) per | (mg) per | | |
| | 0.25 mg | 0.25 mg | | |
| | tablet with | tablet with | | |
| | magnesium | glyceryl | | Reference |
| | sterate as | behenate as | | to |
| Ingredient | lubricant | lubricant | Function | standards |

Tablet core

| | | | | |
|---|---|---|---|---|
| NVS-A | 0.278¹ | 0.278¹ | Drug | Novartis |
| | | | substance | monograph |
| Lactose | 63.897 | 58.797 | Diluent | Ph. Eur./NF |
| monohydrate | | | | |
| Microcrystalline | 17.000 | 17.000 | Diluent | Ph. Eur./NF |
| cellulose/ | | | | |
| Cellulose | | | | |
| microcrystalline | | | | |
| Crospovidone | 2.550 | 2.550 | Disintegrant | Ph. Eur./NF |
| Silica, colloidal | 0.425 | 0.425 | Gliding | Ph. Eur./NF |
| anhydrous/ | | | agent | |
| Colloidal silicon | | | | |
| dioxide | | | | |
| Magnesium | 0.850 | 0 | Lubricant | Ph. Eur./NF |
| stearate | | | | |
| Glyceryl behenate | 0 | 5.950 | Lubricant | |
| Core tablet | 85.000 | 85.000 | | |
| weight | | | | |

Coating

| | | | | |
|---|---|---|---|---|
| Coating premix | 3.400 | 0 | Coating | |
| Opadry AMB | | | agent | |
| white** | | | | |
| Water, purified* | 13.600 | 0 | Solvent | Ph.Eur./ |
| Total film-coated | 88.400 | Not | | USP |
| tablet weight | | applicable | | |

| Coating premix ingredient | Reference to standards |
|---|---|
| Polyvinyl alcohol-part | Ph.Eur./USP |
| hydrolised | |
| Titanium dioxide | Ph.Eur./USP |
| Talc | Ph.Eur./USP |
| Lecithin (soya) | NF |
| Xanthan gum | Ph.Eur./NF |

¹Corresponds to 0.25 mg (e.g. 0.294% w/w) NVS-A base respectively
*Removed during processing
**The qualitative composition of the coating premix is as follows:

1. Magnesium stearate, micronised drug substance X90<8 μm.

2. Glyceryl behenate, micronised drug substance X90<8 μm.

3. Glyceryl behenate, milled drug substance X90=11 μm.

4. Glyceryl behenate, milled drug substance X90=29 μm.

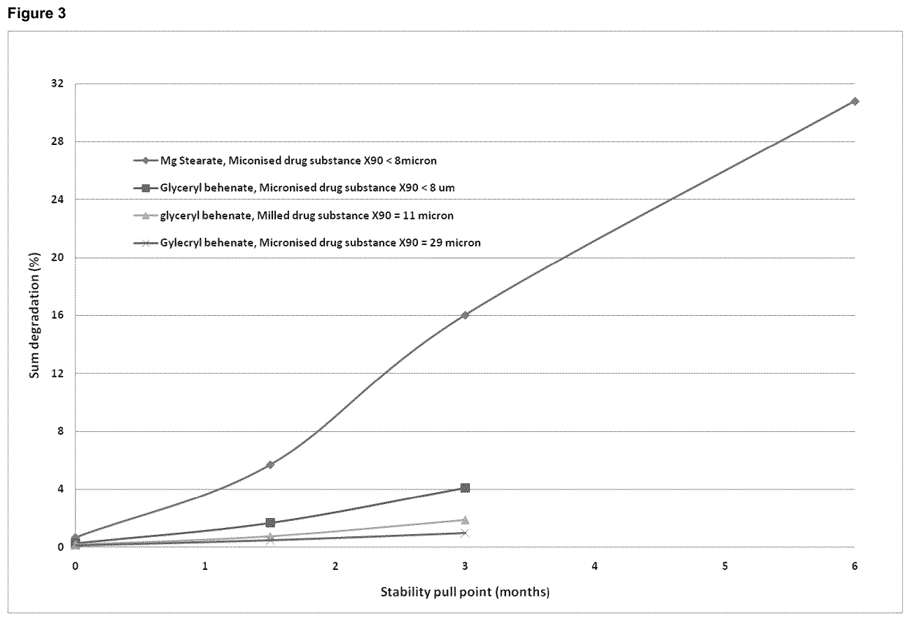

Each of the 4 compositions were stored at 40° C. in 75% relative humidity. The evolution of degradation product for each composition was followed over a period of six months. The results are shown in FIG. 3.
Composition 1 exhibited around 16% degradation product after 3 months of storage.
Composition 2 exhibited around 4% degradation product after 3 months of storage.
Composition 3 exhibited around 2% degradation product after 3 months of storage.
Composition 4 exhibited around 1% degradation product after 3 months of storage.

These results demonstrate that replacing magnesium stearate with glyceryl behenate reduces the sum of degradation product. Furthermore, changing the drug substance quality from micronized (less than 80% crystallinity) to milled (greater than 80% crystallinity) also reduces the sum of degradation product.

1. A solid phase pharmaceutical composition comprising one or more pharmaceutically acceptable excipients and an active pharmaceutical ingredient (“API”) which is a compound of formula A1 or A2 or a pharmacologically acceptable salt, solvate or hydrate thereof, wherein the API is not exposed to a basic compound: 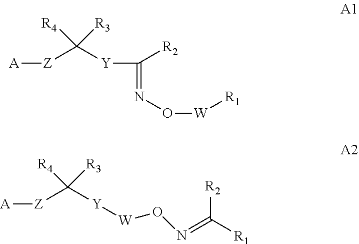
wherein
A is COOR₅, OPO(OR₅)₂, PO(OR₅)₂, SO₂OR₅, POR₅OR₅ or 1H-tetrazol-5-yl, R₅ being H or an ester-forming group;
W is a bond, C₁₋₃alkylene or C₂₋₃alkenylene;
Y is C₆₋₁₀aryl or C₃₋₉heteroaryl, optionally substituted by 1 to 3 radicals selected from halogen, NO₂, C₁₋₆alkyl, C₁₋₆alkoxy; halo-substituted C₁₋₆alkyl and halo-substituted C₁₋₆alkoxy;
Z is chosen from: 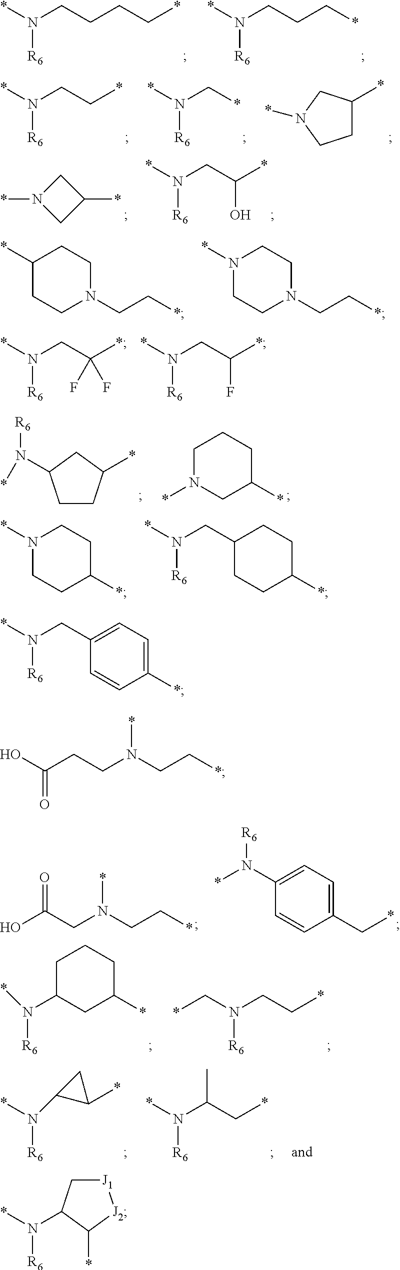
wherein the asterisks of Z indicate the point of attachment between —C(R₃)(R₄)— and A of Formula Ia or Ib, respectively; R₆ is chosen from hydrogen and C₁₋₆alkyl; and J₁ and J₂ are independently methylene or a heteroatom chosen from S, O and NR₅; wherein R₅ is chosen from hydrogen and C₁₋₆alkyl; and any alkylene of Z can be further substituted by one to three radicals chosen from halo, hydroxy, C₁₋₆alkyl; or R₆ can be attached to a carbon atom of Y to form a 5-7 member ring;
R₁ is C₆₋₁₀aryl or C₃₋₉heteroaryl, optionally substituted by C₁₋₆alkyl, C₆₋₁₀aryl, C₆₋₁₀arylC₁₋₄alkyl, C₃₋₉heteroaryl, C₃₋₉heteroarylC₁₋₄alkyl, C₃₋₈cycloalkyl, C₃₋₈cycloalkylC₁₋₄alkyl, C₃₋₈heterocycloalkyl or C₃₋₈heterocycloalkylC₁₋₄alkyl; wherein any aryl, heteroaryl, cycloalkyl or heterocycloalkyl of R₁ may be substituted by 1 to 5 groups selected from halogen, C₁₋₆alkyl, C₁₋₆alkoxy and halo substituted—C₁₋₆alkyl or —C₁₋₆alkoxy;
R₂ is H, C₁₋₆alkyl, halo substituted C₁₋₆alkyl, C₂₋₆alkenyl or C₂₋₆alkynyl; and
each of R₃ or R₄, independently, is H, halogen, OH, C₁₋₆alkyl, C₁₋₆alkoxy or halo substituted C₁₋₆alkyl or C₁₋₆alkoxy.

2. A composition according to claim 1 wherein A is COOH.

3. A composition according to claim 1 wherein the API is 1-{4-[1-(4-cyclohexyl-3-trifluoromethyl-benzyloxyimino)-ethyl]-2-ethyl-benzyl}-azetidine-3-carboxylic acid or a pharmaceutically acceptable salt.

4. A composition according to claim 1 wherein the API is 1-{4-[1-(4-cyclohexyl-3-trifluoromethyl-benzyloxyimino)-ethyl]-2-ethyl-benzyl}-azetidine-3-carboxylic acid or a hemifumarate salt thereof.

5. A composition according to claim 3 wherein the API is in a mixture of materials which is free of basic compounds.

6. A solid phase pharmaceutical composition according to claim 3, wherein the API is in the form of particles having an X90 diameter of at least 8 μm.

7. A composition according to claim 6 wherein the particles have an X90 diameter of from 10 μm to 300 μm.

8. A composition claim 6 which is in unit dosage form and complies with the US Pharmacopeia, European Pharmacopeia and Japanese Pharmacopeia harmonised content uniformity requirements as in force on 1 Jan. 2011.

9. A solid phase pharmaceutical composition according to claim 3, wherein the API has a crystallinity of 80% or more.

10. A tablet comprising a compressed mixture consisting of 1-{4-[1-(4-cyclohexyl-3-trifluoromethyl-benzyloxyimino)-ethyl]-2-ethyl-benzyl}-azetidine-3-carboxylic acid or a pharmaceutically acceptable salt thereof and one or more non-basic excipients, the 1-{4-[1-(4-cyclohexyl-3-trifluoromethyl-benzyloxyimino)-ethyl]-2-ethyl-benzyl}-azetidine-3-carboxylic acid or pharmaceutically acceptable salt being in the form of particles having an X90diameter of from 10 μm to 200 μm.

11. A tablet according to claim 10, wherein the pharamaceutically acceptable salt is a hemifumarate salt.

12. A tablet according to claim 10, wherein said particles are at least 80% crystalline.

13. A tablet according to claim 10, wherein the compressed mixture includes a desiccant and is coated with a moisture barrier.
